(19) United States
(12) Patent Application Publication
(10) Pub. No.: US 2004/0034340 A1
(43) Pub. Date: Feb. 19, 2004

(54) Smart dissector

(75) Inventor: Robert S. Biscup, Vermillion, OH (US)

Correspondence Address: FAY, SHARPE, FAGAN, MINNICH & McKEE, BRIAN E. TURUNG, Seventh Floor, 1100 Superior Avenue, Cleveland, OH, 44114-2579, US

(73) Assignee: Spineco, Inc., an Ohio corporation

(21) Appl. No.: 10/377,430

(22) Filed: Feb. 28, 2003

Related U.S. Application Data
(63) Continuation of application No. 09/687,552, filed on Oct. 13, 2000, now abandoned.
(60) Provisional application No. 60/159,289, filed on Oct. 13, 1999.

(51) Int. Cl. A61B 17/00; A61B 18/18; A61B 18/04

(52) U.S. Cl. 606/1; 606/13; 606/27; 606/45; 606/34

(57) ABSTRACT

A medical cutting device, medical cutting method and/or medical cutting system that detects the type of tissue being cut and/or removed by a cutting device and controls one or more operating conditions of the cutting device based upon the detected type of tissue. The medical cutting device, medical cutting method and/or medical cutting system minimizes or prevents the cutting and/or removal of non-targeted tissue during a medical procedure.

The present invention claims priority on U.S. Provisional Application Serial No. 60/159,289 filed Oct. 13, 1999 entitled “Smart Dissector.”

This invention relates to the art of cutting and/or removing material. The invention is particularly applicable to an apparatus, method and system for cutting and/or removing tissue. However the invention has much broader applications and can be used to cut and/or remove various other materials in a controlled or discriminating manner.

### INCORPORATION BY REFERENCE

The present invention can utilize various types of cutting instruments. These instruments include, but are not limited to, cautery devices, laser devices and ultrasonic devices. U.S. Pat. Nos. 6,126,629; 6,110,162; 6,077,285; 5,733,256; 5,674,235; 5,359,996; 5,151,694; and 4,989,583 disclose and make reference to various types of ultrasonic cutters that can be utilized in the present invention. U.S. Pat. Nos. 6,126,653 and 6,066,126 disclose and make reference to various types of laser cutters that can be utilized in the present invention. U.S. Pat. No. 4,367,744 discloses and makes reference to various types of cautery devices that can be utilized in the present invention. These patents are incorporated by reference herein as background information to explain certain prior cutting device arrangements of which the present invention can utilize. Consequently, the details as to construction and operation of these devices need not be further explained in detail.

### BACKGROUND OF THE INVENTION

During surgery, various electronic devices have been used to cut and/or remove tissue. These devices include cautery devices, laser devices and ultrasonic devices. These devices are utilized in a variety of medical specialties including, but are not limited to, orthopaedics, spinal surgery, neurosurgery, plastic surgery, gynecology, opthalmology, otorhinolaryngology, thoracic- and cardio-thoracic surgery, arthroscopy, invasive, minimally-invasive and general surgery, dermatology, oral surgery, dentistry, podiatric and vetrinary surgery. Ultrasonic devices use ultrasonic energy to emulsify and/or liquify tissue. The emulsified and/or liquified tissue is typically aspirated from the treated site. In a surgical procedure utilizing an ultrasonic device, an incision is typically made in the region of the site where the tissue has to be surgically removed and a narrow cutting tip is inserted into the incision and vibrated ultrasonically to cut and/or remove the desired tissue. The vibrating cutting tip liquifies or emulsifies the tissue and such tissue is aspirated away. Ultrasonic devices can be used to cut and/or remove a variety of tissue. A typical ultrasonic device includes an ultrasonically driven handpiece, an attached cutting tip, and an irrigating sleeve. The handpiece assembly is typically attached to a control console by an electric cable and flexible tubings. Through the electric cable, the console varies the power level transmitted by the handpiece to the attached cutting tip and the flexible tubings supply irrigation fluid to, and draw aspiration fluid from, the tissue treatment area through the handpiece assembly.

Laser devices are used in a variety of surgical applications that involve ablation, vaporization, excision, incision, and coagulation of tissue. The laser device typically includes a metal cannula that encases a fiber optic cable. The fiber optic cable directs laser light to a tissue area resulting in the tissue being cut and/or vaporized. The laser energy typically ranges from about 85 watts of 1064 nm laser energy to about 34 watts of 532 nm laser energy.

Cautery-type or heated-type cutting devices utilize heat to vaporize and remove tissue. The wire cautery portion of the cutting device is connected to an energy source and is designed of a material capable of reaching a suitable temperature for burning away tissue, typically about 500-2000° C.

When using one or more of these cutting devices to cut and/or remove tissue, the cutting device typically encounters different types of tissue during the cutting and/or tissue removal procedure. In many instances, the material which the surgeon desires to remove is irregularly shaped and convoluted and might grow web-like through healthy tissue. The fact that present cutting technologies are indiscriminate in cutting and/or removing tissue results in damage and/or removal of desired, undiseased and/or healthy tissue. For instance, when cancerous tissue is being removed, present cutting devices cut and/or removed healthy tissue cells and structures in addition to the cancerous tissue. This healthy tissue included, but is not limited to, blood vessels, nerve tissue, organ tissue, muscle tissue, etc. In another example, when a herniated or a damaged spinal disk is to be removed to relieve pressure on pinched spinal nerves, the present cutting devices cut and/or removed delicate nerve tissue in addition to the disc material. This indiscriminate cutting and/or removal of desired or healthy tissue resulted in unintentional damage and/or removal of such desired or healthy tissue, damage to surrounding tissue, increased recovery times, and/or increased trauma to the patient.

In view of the present state of technology relating to cutting devices, there is a need for a cutting device or cutting system that can monitor and identify the type of material being cut by the cutting device and control the energy to and/or other parameters of the cutting device to minimize and/or prevent the cutting and/or removal of material is desirable and/or not intended to be cut and/or removed.

### SUMMARY OF THE INVENTION

The present invention relates to the art of cutting and/or removal of material. The invention is particularly applicable to an apparatus, method and system for the controlled cutting and/or removal of tissue during various types of surgical procedures while minimizing or eliminating the cutting and/or removal of tissue not associated with the surgical procedure. However the invention has much broader applications and can be used to cut and/or remove various other material in a controlled manner. These other applications include, but are not limited to, gene splicing, solder or weld metal removal, metal or plastic cleaning, insulation removal, rust removal, carbon deposit removal, adhesive removal, stain removal, and other applications where the goal is for one material is to be discretely cut and/or removed without cutting and/or removing another type of material. Although the present invention will be particularly described with reference to the cutting and/or removal of tissue, it will be appreciated that the below described apparatus, method and system can be similarly utilized in the cutting and/or removal of various other types of materials. Accordingly, the present invention encompasses the application of the disclosed apparatus, method and system to these other material cutting and/or removal applications.

In one aspect of the present invention there is provided a cutting device that includes and/or is used with a material detector. The cutting device can be any type of device that can cut and/or remove tissue. The invention is particularly applicable to electronic cutting devices such as, but not limited to, cautery devices, laser devices and/or ultrasonic devices. The cutting device is used to cut and/or remove various types of tissue during various medical procedures. The material detector is used to monitor, discriminate between, and/or detect the type of tissue being cut by the cutting device. In one embodiment, the material detector obtains tissue information which is used to inform the operator, i.e. surgeon, about the tissue presently being cut and/or removed, and/or informs the operator and/or cutting device system about the tissue that is about to be cut and/or removed. In another embodiment, the material detector is incorporated in the cutting device. In such an arrangement, one device is used to both obtain tissue type information and to cut and/or remove tissue, thereby reducing the number of devices used during the medical procedure, thus simplifying the medical procedure.

In another aspect of the present invention, the material detector sends tissue type information about the tissue being cut and/or removed, and/or about to be cut and/or removed to one or more microprocessors, which one or more microprocessors use such information to partially or fully control one or more components of the cutting device or cutting device system, and/or one or more safety features of the cutting device or cutting device system. The one or more microprocessors can also be designed to receive information from one or more other sources, and then use such information to partially or fully control one or more functions of the cutting device and/or cutting device system. The one or more microprocessors can be hardware and/or software controlled. In one embodiment, the one or more microprocessors are included in the cutting device. In one aspect of this embodiment, at least one microprocessing chip is included in the circuitry of the cutting device. In another embodiment, at least one microprocessor is external to the cutting device. In one aspect of this embodiment, the cutting device transmits and/or receives information to and/or from one or more computers by one or more cables, IR connections, microwave connections, radio wave connection, fiber optic connections, and/or the like. The transmitted and/ore received information can be used to partially or fully control the operation of the cutting device, and/or to provide the user and/or cutting device system information on the progress of the medical procedure, present and/or anticipated operating parameters of the cutting device, tissue type information, and the like. In another embodiment, the one or more computers can include stand alone computers and/or network computers. In still another embodiment, at least one microprocessor is included in the cutting device and at least one microprocessor is external of the cutting device. In this embodiment, certain information processing from and/or to the cutting device, and/or partial or full control of the cutting device is executed internally and externally of the cutting device.

In still another aspect of the present invention, the one or more microprocessors utilized in and/or in conjunction with the cutting device control one or more functions of the cutting device. These functions include, but are not limited to, a) controlling the amount of power and/or energy to the cutting device, b) selecting and/or altering the type of laser or lasers and/or laser wavelength or wavelengths generated by and/or directed to the cutting device, c) selecting and/or altering one or more frequencies of the ultrasonic wavelength generated by and/or directed to the cutting device, d) selecting and/or altering the temperature and/or temperature profile of the tip of the cutting device, e) selecting and/or altering the pulse and/or waveform of the power and/or energy to the cutting device, f) selecting and/or altering the volume and/or flow rate of one or more fluids from the cutting device to the region of cutting and/or removal of tissue, g) selecting and/or altering the type of fluid or fluids directed to the region of cutting and/or removal of tissue, h) selecting and/or altering the volume and/or flow rate of aspiration of fluid or fluids from the region of cutting and/or removal of tissue, i) monitoring one or more functions and/or operating parameters of the cutting device, j) controlling the frequency, amount and/or type of data transfer to and/or from the cutting device, an external monitor, and/or an external microprocessor, k) altering and/or modifying preselected parameters based in view of actual operating parameters, 1) executing one or more search routines to increase the speed of tissue identification, and/or m) controlling one or more automated and/or robotic functions of the medical procedure. As can be appreciated, other control features can be implemented. As a result of such complete or partial microprocessor control, the type of tissue cutting and/or removal procedure can be at least partially electronically controlled. This electronic control frees the operator from having to constantly monitor various parameters of the cutting device during a medical procedure, thus simplifying the medical procedure, increasing the safety and accuracy of the medical procedure, and/or enabling the operator to be more focused on the particular medical procedure.

In yet another aspect of the present invention, the one or more microprocessors utilized in and/or in conjunction with the cutting device monitors and/or activates one or more safety features of the cutting device upon determining a problem during the cutting and/or tissue removal procedure and/or determining that the cutting and/or tissue removal procedure has deviated from one or more selected parameters associated with the cutting and/or tissue removal procedure. The safety features include, but are not limited to, a) activation of audible and/or visual alarms, b) altering or terminating the power to the cutting device, b) increasing or decreasing the volume and/or flow rate of one or more fluids from the cutting device to the region of cutting and/or removal of tissue, c) increasing or decreasing the volume and/or flow rate of aspiration of fluid or fluids from the region of cutting and/or removal of tissue, and/or d) modifying and/or providing suggested modifications to one or more parameters of the medical procedure. As can be appreciated, other safety features can be implemented.

In still yet another aspect of the present invention, the one or more microprocessors utilized in and/or in conjunction with the cutting device compare information received from the material detector on and/or closely adjacent to the cutting device to one or more tissue databases to determine the type tissue being cut and/or removed by the cutting device, and/or about to be cut and/or removed by the cutting device. The one or more tissue databases can be hardwired in the one or more microprocessors and/or be stored on a temporary media such as, but not limited to, RAM, floppy disk, hard drive, CD, DVD, ZIP drive, and/or the like. The one or more microprocessors, after making a determination of the type of tissue being cut and/or removed, and/or after making a determination of the type of tissue not being cut and/or removed, utilize such information to control or partially control one or more function of the cutting device or cutting device system and/or one or more safety features of the cutting device or cutting device system. In one embodiment, the one or more tissue databases only include information about the tissue to be cut and/or removed. In one aspect of this embodiment, the one or more microprocessors determine whether or not the particular tissue to be cut and/or removed is indeed being cut or removed. When another type of tissue information is transmitted by the material detector to the one or more microprocessors, the one or more microprocessors make the determination that such tissue is a tissue other than the tissue to be cut and/or removed. In another aspect of this embodiment, based upon preselected control settings, the one or more microprocessors implement one or more control settings when the desired tissue is determined as being cut and/or removed by the cutting device, and implements other controls when an unidentified tissue is determined as being cut and/or removed by the cutting device. In another embodiment, the one or more tissue databases include information on the tissue to be cut and/or removed and on one or more other tissues that are near and/or adjacent to the tissue to be cut and/or removed. In one aspect of the embodiment, the one or more microprocessors not only determine the tissue to be cut and/or removed, but determine other tissues that are encountered which are near and/or adjacent to the tissue to be cut and/or removed. In another aspect of this embodiment, the one or more microprocessors partially or fully control one or more functions of the cutting device or cutting device system based upon the various tissues encountered during the medical procedure. Alternatively or in addition to, the one or more microprocessors can activate one or more safety features of the cutting device or cutting device system when a particular tissue is detected as being cut and/or removed. In still another embodiment, the one or more tissue databases include one or more type of physiologic tissue information about each type of tissue. Such information on the tissue includes, but is not limited to, a) electrical conduction and/or resistance of the tissue, b) chemical composition of the tissue, c) heating and/or cooling rates of the tissue, d) tissue density, e) tissue viscosity, f) sound and/or electromagnetic wave reflective and/or refractive properties of the tissue, g) eddy current properties of the tissue, h) tissue color, and/or i) tissue temperature. In one aspect of this embodiment, the database includes tissue properties obtained from sampling several similar tissues from a variety of sources. In another aspect of this embodiment, the database includes tissue properties obtained from statistical modeling of the tissue. In yet another aspect of this embodiment, the tissue database is updated to include additional tissue sources and/or additional tissue parameters. In still another aspect of this embodiment, the tissue information obtained by the one or more material detectors is loaded into the material database to update the database during the medical procedure and/or increase the speed at which the tissue being cut and/or removed is identified by the one or more microprocessors. In still yet another aspect of this embodiment, a sample of the tissue that is to be cut and/or removed during the medical procedure is analyzed and/or loaded into the one or more tissue databases prior to conducting the surgical procedure. The tissue sample can be taken by a biopsy or other technique. In addition to taking a sample of the tissue to be cut or removed, surrounding tissue can also be taken so as to analyze and/or load such information about the tissues into the one or more tissue databases. As can be appreciated, such sampling increases the tissue information in the tissue database thereby increasing the accuracy of tissue type determination by the one or more microprocessors. In still yet another aspect of this embodiment, the data in the one or more tissue databases is shared with other tissue databases so as to further increase the tissue information in the one or more tissue databases. In a further aspect of this embodiment, the one or more tissue databases include data structures that minimize the time necessary to search the data on the database to obtain a tissue match. In still a further aspect of this embodiment, the database can include one or more subroutines which increases the speed at which a tissue match is obtained. Such subroutines can include the ignoring of blocks of data in the database from a particular tissue search based upon certain tissue data obtained from the one or more material detectors. This type of subroutine when used in conjunction with a well defined data structure in the database will significantly reduce the time necessary to identify a particular tissue. Other subroutines can alternatively or also be used that involve simple or complex searching routines (i.e interpolation, fuzzy logic, etc.). As a result, near real time tissue identification during the medical procedure is realized.

In a further aspect of the present invention, the material detector obtains chemical, electrical and/or mechanical information about the tissue being cut and/or removed, and/or about to be cut and/or removed by one or more techniques, and transmits such information to the one or more microprocessors to enable the one or more microprocessors to identify the tissue type. The tissue chemical, electrical and/or mechanical information can be obtained from various techniques that include, but are not limited to, a) electrical conduction and/or resistance measurements, b) chemical composition measurements, c) rate of heating and/or cooling measurements, d) density measurements, e) viscosity measurements, f) sound and/or electromagnetic wave reflective and/or refractive measurements, g) eddy current measurements, h) tissue color measurements; and/or i) tissue temperature measurements. In one embodiment, the tissue type measurement corresponds to an electrical characteristic of the tissue so that the tissue identification can be quickly made while the cutting device is in the process of cutting and/or removing tissue. In one aspect of this embodiment, the tissue type measurement includes the electrical conduction and/or resistance of the tissue.

In still a further aspect of the present invention, a system for cutting and/or removing tissue is provided which system utilizes a cutting device and a material detector. The system is designed to integrate a cutting device with a tissue recognition system to enable an operator of the cutting device to distinguish and/or differentiate among various types of tissue during the cutting and/or tissue removal procedure. The material detector is used to detect one or more distinct physiologic characteristics of the tissue being cut or removed and/or tissue about to be cut and/or removed. The detected one or more distinct physiologic characteristics are transmitted to one or more microprocessors and compared with one or more tissue databases containing one or more distinct physiologic tissue characteristics. The one or more microprocessors, after comparing the measured distinct physiologic tissue characteristics to the distinct physiologic tissue characteristics in the one or more tissue databases, make a determination of the type of tissue which has been cut or removed and/or tissue about to be cut and/or removed. Once the tissue has been identified, this information can be used to control the operation of the cutting device. In one particular system, the tissue information is used to control the power to the cutting device so as to minimize or prevent the cutting and/or removal of tissue that is not supposed to be cut and/or removed during the medical procedure. As a result, this particular system is designed to cut and/or remove tissue discriminately during the medical procedure. Consequently, only the targeted tissue is cut or removed and the other types of tissue are not cut and/or removed by the cutting device. Such a cutting system is termed a “smart dissection device.” The “smart dissection device” can be designed for use with manual procedures, designed to be used with microsurgical techniques and/or designed to be used in robotic-controlled surgical techniques.

In summary, the present invention involves the linking of a surgical cutting device (i.e. cautery devices, laser devices and/or ultrasonic devices) to a smart tissue detection system or circuit which tissue detection system or circuit can identify and/or differentiate between various types of tissue (i.e. organs, blood vessels, nerves, bones, muscles, fat, lymph nodes, lymph ducts, brain tissue), various sub-types of a specific tissue (i.e. diseased tissue verses undiseased tissue), and/or various portions of cellular or molecular structures (i.e. golgi bodies verses mitochondrial structures, helical verses non-helical chromosomes, etc.) by means of distinct physiologic characteristics of the various types of tissue, sub-types, cells and/or molecules. The tissue detection system or circuit includes one or more tissue databases which contain one or more tissue physiologic characteristics. The tissue data that is stored in the one or more tissue databases can be obtained from one or more sampled tissues. One specific implementation of the invention involves the use of an ultrasonic cutting device which can emulsify, liquify and/or dissolve tissue. The ultrasonic device includes a material detector which detects and transmits one or more detected tissue physiologic characteristics to a microprocessor. The microprocessor compares the detected tissue physiologic characteristics to tissue physiologic characteristics contained in a tissue database and determines the type of tissue at or near the ultrasonic cutting device. Based on predetermined and/or preset criteria, the microprocessor controls the operation of the ultrasonic cutting device. Such control includes, but is not limited to, a) maintaining the present operating conditions of the ultrasonic cutting device when the targeted tissue to be cut and/or removed is detected, b) reducing or terminating power to the ultrasonic cutting device when non-targeted tissue is being cut and/or removed or about to be cut and/or removed, thereby minimizing or preventing the removal of such non-targeted tissue. Such a cutting device system creates an enhanced surgical system that spares good tissue and cuts and/or removes unwanted tissue. This “smart dissection system” will significantly advance surgical techniques by enabling efficient tissue dissection while reducing or eliminating adverse tissue removal. The “smart dissection system” can be sized and configured for use in large units which can be used manually without magnification or sized and configured for use with a microscopic probe used in microsurgery and microsurgical dissection or even smaller. The “smart dissection system” can include the use of a feedback circuit which enables data to be transmitted from the ultrasonic cutting device to the microprocessor, which microprocessor determines the tissue type and based upon the predetermined or preset criteria for the medical procedure, transmits control information to the ultrasonic cutting device. As can be appreciated, the “smart dissection system” enables the cutting and/or removal of tissue for simple and sophisticated medical procedures. For example, the “smart dissection system” can be used to remove cancerous cells around a complex array of nerves and blood vessels while leaving the nerves and blood vessels intact and unharmed. As can be appreciated, the “smart dissecting system” has many applications other than surgical applications. For instance, the “smart dissection system” can be used to remove unwanted materials (i.e. carbon, mineral deposits) from valves, or removing solder and/or impurities from circuit boards and/or computer chips. In theory, the “smart dissection system” could also be used in various types of genetic splicing applications.

The principal object of the present invention is to provide an improved apparatus, method and system for discriminately cutting and/or removing tissue.

Another object of the present invention is to provide an improved apparatus, method and system for cutting and/or removing undesired or unhealthy tissue while preventing or minimizing the removal or damage to desired or healthy tissue.

Still another object of the present invention is to provide an improved apparatus, method and system for cutting and/or removing a variety of tissues.

Yet another object of the present invention is to provide an improved apparatus, method and system for cutting and/or removing tissue in an efficient and effective manner.

Still yet another object of the present invention is to provide an improved apparatus, method and system of cutting and/or removing tissue in a manner that reduces patient trauma.

A further object of the present invention is to provide an improved apparatus, method and system for cutting and/or removing tissue which may be easily used and performed by a surgeon of any skill.

Still a further object of the present invention is to provide an improved apparatus, method and system for cutting and/or removing tissue which identifies one or more tissues during the cutting and/or removal procedure.

Yet a further object of the present invention is to provide an improved apparatus, method and system for cutting and/or removing tissue which utilizes a tissue database to identify tissue being cut and/or removed and/or about to be cut and/or removed.

Still yet a further object of the present invention is to provide an improved apparatus, method and system for cutting and/or removing tissue which utilizes a tissue database that includes one or more tissue characteristics for each tissue in the database.

Another object of the present invention is to provide an improved apparatus, method and system for cutting and/or removing tissue which utilizes a feedback circuit and tissue identification information to control one or more functions of the cutting device.

Still another object of the present invention is to provide an improved apparatus, method and system for cutting and/or removing tissue which provides information to the operator relating to the type of tissue being cut and/or removed and/or about to be cut and/or removed, and one or more operating conditions of the cutting device.

Yet another object of the present invention is to provide an improved apparatus, method and system for cutting and/or removing tissue which enables the tissue database to be modified, updated, networked, and the like.

Still yet another object of the present invention is to provide an improved apparatus, method and system for cutting and/or removing tissue which activates and/or implements one or more safety functions when one or more parameters of the medical procedure are deviated therefrom.

These and other objects and advantages will become apparent to those skilled in the art upon the reading and following of this description taken together with the accompanied drawing.

### BRIEF DESCRIPTION OF THE DRAWINGS

Reference may now be made to the drawings, which illustrate various embodiments that the invention may take in physical form and in certain parts and arrangements of parts wherein;

FIG. 1 is a block diagram of one embodiment of the cutting device system of the present invention; and

FIG. 2 is an enlarged partial perspective view of the end of the cutting device showing the material detector on the end of the cutting device.

### DETAILED DESCRIPTION OF THE PREFERRED EMBODIMENTS

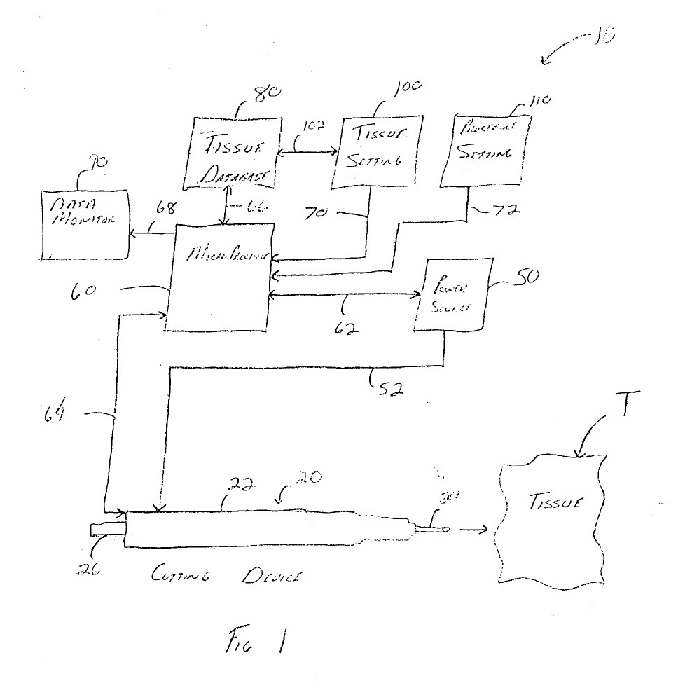

Referring now to the drawings, wherein the showings are for the purpose of illustrating the preferred embodiments of the invention only and not for the purpose of limiting the same, in FIG. 1 there is shown a cutting device system 10 which includes an information communication system to provide information to an operator and receive information from the operator which information is representative of operating parameters of a cutting device 20 and environment the cutting device is being used therein.

Cutting device 20 is one of a number of electronic cutting devices that can be used (i.e. cautery devices, laser devices and ultrasonic devices). For purposes of specific illustration only, a typical ultrasonic surgical device will be described in detail. However, the substitution of other electronic cutting devices for the ultrasonic surgical device is readily apparent from the invention. Cutting device 20, when being an ultrasonic cutting device, includes an ultrasonically driven handpiece 22, an attached cutting tip 24, and an irrigating sleeve 26. Irrigating sleeve 26 is connected to tubing, not shown, which provides one or more fluids to the cutting device and/or removes fluid from the cutting device. The handpiece 22 is connected to an external power source by power cable 52; however, the power source for the cutting device can be integrated in handpiece 22. The power source provides the power level transmitted by handpiece 22 to attached cutting tip 24. The tubings supply irrigation fluid to and/or draw aspiration fluid from tissue T which is being cut and/or removed by the cutting device. The handpiece includes a resonating bar or horn that is attached to one or more piezoelectric crystals. The crystals supply the required ultrasonic vibration needed to drive both the horn and the attached cutting tip 24. A frequency of between 0.5 Hz and 200 Hz is generally used to emulsify and/or liquify the tissue; however, other frequencies can be used. The frequency is selected depending on the type of tissue to be cut, emulsified, and/or liquified. Generally, a lower frequency with a higher pulse rate is used to cut, emulsify, and/or liquify relatively harder tissue, and a higher frequency and lower pulse rate is used to cut, emulsify, and/or liquify softer tissue. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip 24. The irrigation sleeve also has an internally threaded bore that is screwed onto the external threads of the nosecone. The cutting tip 24 is adjusted so that the tip projects only a predetermined amount past the open end of the irrigating sleeve.

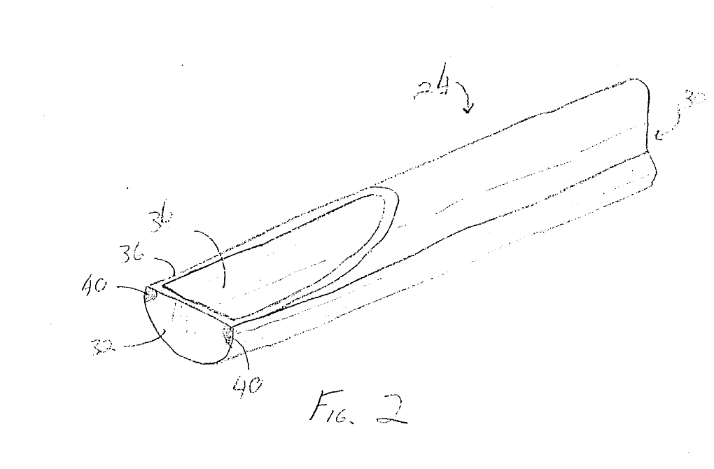

FIG. 2 illustrates an enlarged end portion of cutting tip 24. The end of cutting tip 24 includes a hollow tube which has a blunt or rounded distal end 32. An opening 34 is cut at an angle into the side of the hollow tube. A sharp edge 36 is formed in the hollow tube around the periphery of opening 34. Positioned on distal end 32 are two tissue detectors 40. The tissue detectors are designed to obtain tissue type information relating to the type of tissue at and/or closely adjacent to distal end 32 of cutting tip 24. The configuration of the cutting tip is designed to easily and efficiently cut and/or remove tissue T when the cutting tip is ultrasonically vibrated. As can be appreciated, many other types of cutting tip designs can be used in the present invention. A few of these designs are illustrated and described in the prior art which is incorporated herein by reference.

In use, the end of the cutting tip is inserted into a small incision. The cutting tip is ultrasonically vibrated along its longitudinal axis by the crystal-driven ultrasonic horn thereby causing emulsification and/or liquification of the targeted tissue in situ. The hollow bore of the cutting tip communicates with the aspiration line connected to the handpiece. A reduced pressure or vacuum source draws or aspirates the emulsified and/or liquified tissue from the surgical site through opening 34 of cutting tip 24. The aspiration of the emulsified and/or liquified tissue is generally aided by a flushing solution or irrigant that is injected into the surgical site, typically through a small annular gap at or adjacent to the end of cutting tip 24. The flushing solution or irrigant can be heated prior to being injected into the surgical site. The operation of the cutting device during the medical procedure can be performed by hand and/or by one or more robotic and/or automated controls. The use of general automated and/or robotic control techniques are know in the art, thus will not be particularly described herein.

Referring again to FIG. 1, cutting device system 10 is designed to control one or more functions of cutting device 20 during a particular medical procedure. As shown in FIG. 1, the power to cutting device 20 is supplied by power source 50 through power cable 52. As illustrated, the power source is external to the cutting device. As can be appreciated, the power source can be fully or partially located in the cutting device. The power source is controlled by microprocessor 60. Microprocessor 60 can be in the form of a hardwired circuit, one or more stand alone computers, and/or one or more networked computers. As illustrated, the microprocessor is external to the cutting device. As can be appreciated, the microprocessor can be fully or partially located in the cutting device. Typically, microprocessor 60 includes a stand alone computer or networked computer. Microprocessor 60 is connected to several data inputs and outputs in the cutting device system. As shown in FIG. 1, microprocessor 60 transmits and receives data from power source 50 via power control cable 62. Microprocessor 60 also transmits and receives data from cutting device 20 via device control cable 64. Microprocessor 60 also transmits and receives data from tissue database 80 via database cable 66. Microprocessor 60 transmits data to data monitor 90 via monitor cable 68. Microprocessor 60 receives data from tissue setting 100 via tissue type cable 70. Data is also transmitted between tissue setting 100 and tissue database 80 via database setting cable 102. Microprocessor 60 also receives data from procedure setting 110 via procedure type cable 72. As can be appreciated, the data cables described above can be substituted for fiber optic cables, IR connections, radio wave, microwave connections, and the like. In addition, one or more of the data cables can be part of a hardwired circuit. As illustrated, the data monitor, tissue database, tissue setting and procedure setting are external to the cutting device. As can be appreciated, one or more of these components can be fully or partially integrated on or in the cutting device.

Microprocessor 60 is designed to function as the central control for the cutting device system. The microprocessor partially or fully controls the operation of cutting device 20 based upon preselected inputs and actual data received from the cutting device during the cutting and/or removal of tissue T. The operation of the microprocessor can be hardware and/or software controlled. When the microprocessor is or includes one or more computers, the microprocessor is at least partially software controlled.

The data monitor 90 typically includes one or more displays. The data monitor displays one or more procedure settings, tissue type settings, determined tissue type, control parameters of the cutting device and/or operating parameters of the cutting device. The data monitor can also include audible and/or visual alarms to indicated when one or more parameters of the medical procedure have exceeded certain parameters and/or deviated from preselected settings. The data monitor can also function as a user interface by which the user (such as a surgeon, medical technician or assistant) receives information representative of the various operating parameters of the cutting device and enables the user to manually modify one or more settings and/or operating parameters of the medical procedure. As a result, the user is able to reprogram or select from the operating parameters stored in the microprocessor. One such data monitor includes a touch display panel which enables the user to manually interface with one or more parameters associated with the medical procedure.

During operation, the user selects the type of medical procedure to be performed on procedure setting 110. The selection of the procedure can be as simple as selecting a single procedure, which in turn sends a set of preselected parameters to microprocessor 60 via procedure type cable 72. Alternatively, the selection of a particular procedure causes one or more preselected parameters and/or parameter data fields to be displayed thereby allowing the user to input and/or modify the data for one or more of the parameters. The user also selects the type of tissue that is to be cut and/or removed during the medical procedure on tissue setting 100. This tissue information is transmitted to microprocessor 60 via tissue type cable 70. Tissue setting 100 can also enable the user to select other types of tissue that may be encountered during the medical procedure. Tissue setting 100 can also be used by the user to check the types of tissues and associated tissue parameters in the tissue database and, if desired, enable the user to add information to the tissue database and/or modify information in the tissue database. Such an operation allows the user to update and/or include additional tissue data in the tissue database and allows the user to make special modifications to the tissue data for one or more tissues that will be, or is anticipated to be, encountered during the medical procedure. As can be appreciated, the selection of the medical procedure on procedure setting 110 can include a parameter relating to the type of tissue to be cut and/or removed during the medical procedure. Alternatively, the selection of the type of tissue to be cut and/or removed on tissue setting 100 can result in a particular procedure setting being automatically selected. Once the tissue settings and procedure settings are selected and loaded into the microprocessor, one or more of these settings are displayed on data monitor 90. The display of the settings enable the user to confirm that the settings have been correctly selected.

Once the settings have been confirmed by the user and loaded into, and/or accessed by, the microprocessor, the microprocessor runs various hardware and/or software algorithms based upon the selected parameters to produce the initial control settings for the cutting device. For instance, when the cutting device is an ultrasonic cutter, the microprocessor selects the initial ultrasonic frequency, the pulse rate and/or intensity of the ultrasonic frequency. When the cutting device is a laser cutter, the microprocessor selects the laser wavelength, the pulse rate and/or intensity of the laser wavelength. When the cutting device is a cautery cutter, the microprocessor selects the energy level and/or temperature of the cautery wire. As can be appreciated, the microprocessor can select other initial settings for the cutting device including, but not limited to, a) the type, flowrate and/or temperature of the irrigant through the cutting device, and/or b) the flowrate of aspirated fluid from the surgical site.

Prior to the user activating the cutting device, the user typically creates an incision in the patient to enable the user to position the cutting tip of the cutting device near or in the targeted tissue to be cut and/or removed when the targeted tissue is encircled by other types of tissue. However, the cutting device can be used to cut tissue until the cutting tip reaches the targeted tissue. The cutting or the incision and/or positioning of the cutting tip of the cutting device near or in the targeted tissue can be performed manually, by automated controls, and/or by robotic controls.

During the cutting and/or tissue removal procedure, the user activates cutting device 20. Microprocessor 60, upon receiving a data signal that the cutting device has been activated, controls information to power source 50 to cause power source 50 to deliver to cutting device 20 the desired amount of power as determined by the preselected settings. In addition, microprocessor 60 can send control instructions to the cutting device to further control the operation of the cutting device. As can be appreciated, the user can modify one or more parameters of the medical procedure during the tissue cutting and/or removal procedure. As cutting device 20 cuts and/or removes tissue T, various types of information concerning the operation of the cutting device and/or tissue information encountered and/or about to be encountered by the cutting device are transmitted to the microprocessor. The microprocessor uses this information to maintain and/or alter one or more control and/or operating parameters of the cutting device. Typically, the cutting device includes one or more sensors to detect one or more actual operating parameters of the cutting device and to send such information to the microprocessor. These sensors include, but are not limited to, temperature sensors, energy or power sensors, light sensors, frequency sensors, flowrate sensors, and the like.

In addition to the one or more operating parameters of the cutting device received by the microprocessor, tissue information relating to tissue being cut and/or removed, and/or about to be cut and/or removed is transmitted to the microprocessor. The tissue information is obtained by tissue detectors 40 located at the end of cutting tip 24. In one embodiment, the tissue detectors include electrodes which measure electrical resistance of the tissue at or closely adjacent to the end of cutting tip 24. This electrical resistance data is transmitted to microprocessor 60 wherein the data is compared with electrical resistance data of various tissues in tissue database 80 to identify the particular tissue being detected by the tissue detectors. The determined tissue information and/or measured tissue property can be displayed on data monitor 90 along with one or more other operating parameters of the cutting device or cutting system. When the tissue type determined by the microprocessor matches the tissue type preselected to be cut and/or removed during the medical procedure, the microprocessor sends signals to the power supply to maintain power to the cutting device. However, when the microprocessor determines that a tissue being cut and/or removed by the cutting device is not a tissue selected to be cut and/or removed, the microprocessor sends a control signal to the power source to cause the power source to reduce and/or terminate the power to the cutting device. As a result, non-targeted tissue that is encountered during the medical procedure is not cut and/or removed or essentially not cut or removed by the cutting device. In addition to the power being terminated or reduced, the microprocessor can send a signal to the data monitor to activate an alarm indicating a deviation in a preselected tissue type parameter. The user, upon receiving the alarm, and/or noticing and/or being informed of a power reduction or termination to the cutting device, can take the appropriate corrective action. Such action can include, but is not limited to, a) the user redirecting the cutting tip to cause the cutting tip to reengage the targeted tissue where, upon detection of the targeted tissue, the microprocessor causes power to once again be directed to the cutting device, b) the user overriding the control parameters and/or modifying the control parameters to continue tissue cutting and/or removal, or c) the user terminating the medical procedure and removing the cutting device from the patient. Alternatively, if the tissue cutting and/or removal procedure is automated, the control circuit, based upon preselected parameters, can take the appropriate corrective action. Such action can include, but is not limited to, a) redirecting the cutting tip so cause the cutting tip to reengage the targeted tissue where upon detection of the targeted tissue, the microprocessor causes power to once again be directed to the cutting device, b) overriding the control parameters and/or modifying the control parameters to continue tissue cutting and/or removal, or c) terminating the medical procedure and removing the cutting device from the patient. As can be appreciated, other manual or automated actions can be taken.

The invention has been described with reference to a preferred embodiment and alternates thereof. It is believed that many modifications and alterations to the embodiments disclosed will readily suggest itself to the those skilled in the art upon reading and understanding the detailed description of the invention. It is intended to include all such modifications and alterations insofar as they come within the scope of the present invention.

I claim:

1. A medical cutting device comprising:
an power source;
a cutting device having at least one cutting tip that receives power from the power source and directs power at tissue positioned at or closely adjacent to the cutting tip to cause the tissue to be cut, liquified and/or emulsified; and
a material detector to obtain tissue characteristic information of said tissue at, and/or closely adjacent to, said cutting tip.

2. The medical cutting device as defined in claim 1, including a microprocessor, said microprocessor receiving tissue information from said material detector and controlling at least one function of said cutting device as a function of said received tissue information.

3. The medical cutting device as defined in claim 2, wherein said controlling at least one function of said cutting device includes controlling the power to said cutting tip.

4. The medical cutting device as defined in claim 2, wherein said microprocessor controlling at least one safety feature of said cutting device as a function of said received tissue information.

5. The medical device as defined in claim 2, including a tissue database, said microprocessor comparing information in said tissue database to said tissue information received from said material detector to identify said tissue at and/or closely adjacent to said cutting tip.

6. The medical device as defined in claim 1, wherein said tissue characteristic information includes at least one physiologic characteristic of said tissue.

7. The medical device as defined in claim 6, wherein said at least one physiologic characteristics includes a characteristic selected from the group consisting of electrical conduction of said tissue, electrical resistance of said tissue, chemical composition of said tissue, heating rates of said tissue, cooling rate of said tissue, density of said tissue, viscosity of said tissue, sound penetration properties of said tissue, light penetration properties of said tissue, electromagnetic wave reflective properties of said tissue, electromagnetic wave refractive properties of said tissue, eddy current properties of said tissue, color of said tissue, temperature of said tissue, and combinations thereof.

8. The medical device as defined in claim 6, wherein said physiologic characteristics of at least one tissue can be updated in said database.

9. The medical device as defined in claim 1, wherein said material detector is connect to said cutting tip.

10. The medical device as defined in claim 1, wherein said power source directs power to said cutting tip in the form of heat, electricity, laser light, ultrasonic waves, electromagnetic waves, and combinations thereof.

11. The medical device as defined in claim 1, wherein said cutting tip is connected to a cutter selected from the group consisting of a cautery cutter, a laser cutter, an ultrasonic cutter, and combinations thereof.

12. A tissue identification device comprising:
a tissue probe having an end portion;
at least one material detector portioned at least closely adjacent to said end portion of said tissue probe, said at least one material detector obtaining information on at least physiologic tissue characteristic from a tissue positioned at and/or closely adjacent to said end portion of said tissue probe when said end portion is positioned at least closely adjacent to said tissue;
a tissue database that includes physiologic tissue data for at least one type of tissue; and
a microprocessor that compares physiologic tissue characteristics obtained by said at least one material detector to physiologic tissue data in said tissue database.

13. The tissue identification device as defined in claim 12, wherein said tissue database includes physiologic tissue data for a plurality of tissues.

14. The tissue identification device as defined in claim 12, wherein said tissue database includes a plurality of physiologic tissue data for at least one tissue in said tissue database.

15. The tissue identification device as defined in claim 12, wherein said tissue database can be updated.

16. The tissue identification device as defined in claim 12, wherein said tissue probe includes a cutting device.

17. A system of discretely removing tissue comprising:
an electronic cutting device having at least one cutting tip, said cutting tip designed to cut, liquify and/or emulsify tissue positioned at or closely adjacent to the cutting tip;
a power source that at least partially provides power to said electronic cutting device;
a material detector that obtains information on physiologic tissue characteristics of tissue positioned at and/or closely adjacent to said cutting tip;
a tissue database that includes physiologic tissue characteristic data for at least one type of tissue; and
a microprocessor, said microprocessor comparing at least one physiologic tissue characteristic obtained by said at least one material detector to physiologic tissue characteristic data in said tissue database and to attempt to identify at least one tissue positioned at and/or closely adjacent to said cutting tip, said microprocessor controlling at least one function of said electronic cutting device as a function of the results of the tissue comparison.

18. The system as defined in claim 17, wherein said at least one function controlled by said microprocessor includes the amount of power to said electronic cutting device.

19. The system as defined in claim 17, including a data monitor to display at least one preset and/or actual operating parameter of said electronic cutting device.

20. A method for discretely removing tissue comprising:
a) providing an electronic cutting device adapted to cut, liquify and/or emulsify tissue;
b) directing said electronic cutting device toward a target tissue;
c) obtaining physiologic tissue characteristic information for at least one tissue positioned at and/or closely adjacent to said electronic cutting device; and
d) controlling at least one operating condition of said electronic cutting device as a function of the obtained physiologic tissue information.

21. The method as defined in claim 20, wherein said at least one operating condition includes power to said electronic cutting device.

22. The method as defined in claim 20, including the step of identifying a type of tissue based at least partially on said physiologic tissue characteristic information.

23. The method as defined in claim 22, wherein said step of identifying includes a tissue database, said tissue database including physiologic tissue characteristic data for at least one type of tissue.

24. The method as defined in claim 23, wherein said step of identifying includes a microprocessor designed to compare said obtained physiologic tissue characteristic information to said physiologic tissue characteristic data in said tissue database to attempt to identify at least one tissue.

25. The method as defined in claim 20, wherein said step of obtaining physiologic tissue characteristic information includes at least a material detector positioned at least closely adjacent to said electronic cutting device.
